(19) United States
(12) Patent Application Publication
(10) Pub. No.: US 2006/0147506 A1
(43) Pub. Date: Jul. 6, 2006

(54) Foam with germproof and mildew-proof effects

(76) Inventors: Peng-Yi Kuo, Minsyong Township (TW); Hsiung-Tsai Sun, Sinjhuang City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/028,002

(22) Filed: Jan. 4, 2005

(51) Int. Cl. A61K 9/70; A61K 36/37; A61K 36/232

(52) U.S. Cl. 424/443; 424/759; 424/725

(57) ABSTRACT

The present invention relates to foam with the germproof and mildew-proof effects. To produce such foam, Chinese herbaceous extracts are prepared by extracting from angelica-laxiflora, ranunculacese-clematis chinensis, tripterygium wilfordli hook, Atractylodes lancea(Thunb)DC, talc powder, alunite, Cnidium monnieri(L.)Cuss and hydnocarpus anthelmintica P. These extracts are then blended with plastic or rubber material and brought to a foaming treatment. Accordingly, plastic or rubber products with germproof and mildew-proof effects are manufactured, and the effects can be lasted.

### BACKGROUND OF THE INVENTION

1. Field of the Invention

The present invention relates to foam with germproof and mildew-proof effects. To produce such foam, the Chinese herbaceous extracts with germproof and mildew-proof effects are added in plastics or rubbers. Accordingly, daily-used articles made from the foam may possess germproof and mildew-proof effects.

2. Description of the Related Prior Arts

Conventionally, the Chinese herbaceous extracts with germproof and mildew-proof effects are produced in the forms of paste or powder and contained in cans. The paste or powder is usually utilized by spraying or applying it on the plastic or rubber articles which need to be germproof and mildew-proof.

However, the manner of spraying powder or applying paste on daily-used articles is not convenient for users.

Moreover, when used for a period of time, these articles might lose the mildew-proof and germproof effects due to volatility or friction, since the layer with such effects is merely attached on surfaces thereof.

Therefore, it is desired to provide an option for users, so that the Chinese herbaceous extracts with germproof and mildew-proof effects can be conveniently utilized in daily lives.

### SUMMARY OF THE INVENTION

The object of the present invention is to provide foam with germproof and mildew-proof effects, so that users' health can be improved and the environments can be cleaner and more comfortable.

In order to achieve the above object, Chinese herbaceous extracts with germproof and mildew-proof effects are added into a plastic or rubber material in predetermined ratios. The Chinese herbaceous extracts primarily extracted from angelica-laxiflora, ranunculacese-clematis chinensis, tripterygium wilfordli hook, Atractylodes lancea(Thunb)DC, Cnidium monnieri(L.)Cuss and hydnocarpus anthelmintica P, and them ground with talc powder and alunite. By blending the Chinese herbaceous extracts with the plastic or rubber material, plastic or rubber products with germproof and mildew-proof effects can be obtained. The products with germproof and mildew-proof effects may be applied to daily life, and thus improve the users' health and living quality.

Pharmacological effects, pathological effects, chemical components and contents of the components used in the present invention are as follows:
(1) Angelica-Laxiflora:

pharmacological effects: relieving pain, reducing inflammation, mildew-proofing;

pathological effects: dispelling wind, drying dampness, relieving pain due to arthralgia, preventing dermatoses;

chemical components: angelica pubescens maxim, angelica acid angelol, angelicone, bergapten;

content in the herbs: 15 wt. %;

ratio of angelica-laxiflora to the foaming material: 0.0075:1.
(2) Ranunculacese-Clematis Chinensis

pharmacological effects: anodyne, stopping itch, treating pruritus;

pathological effects: dispelling wind, drying dampness, passing meridians, relieving pain;

chemical components: anemonin, hederagenin-anemonol, ponin (iresenin), sitosterin, myristic acid, linolenic acid, oleic acid;

content in the herbs: 12 wt. %;

ratio of ranunculacese-clematis chinensis to the foaming material: 0.006:1.
(3) Tripterygium Wilfordli Hook

pharmacological effects: anti-inflammation, immunosuppression;

pathological effects: reducing swelling or edema, relieving pain, treating pruritus, treating skin itch, treating skin spots, destroying vermifuge;

chemical components: wilfordine, witforgine, wilfortrine, tripdiolide, triptonide, hypolide, dulcitol;

content in the herbs: 10 wt. %;

ratio of tripterygium wilfordli hook to the foaming material: 0.005:1.
(4) Atractylodes Lancea(Thunb)DC

pharmacological effects: destroying bacteria, inhibiting growth of mold or fungus as formalin;

pathological effects: dispelling wind, drying dampness, inhibiting growth of bacteria, germproof, treating eczema, treating urticaria;

chemical components: atractylol, hinesol, β-eudesmol, β-selinene, acractylodine, 3,5-dimethoxy-lucosyloxy phenylpropenyl, alcohol;

content in the herbs: 15 wt. %;

ratio of Atractylodes lancea(Thunb)DC to the foaming material: 0.0075:1.
(5) Talc Powder

pharmacological effects: protecting mucous membranes of skin, absorbing chemical stimulants or toxicants;

pathological effects: treating eczema, treating acne;

chemical components: Mg₃(Si₄O₁₀(OH)₂)Al₂O₃;

content in the herbs: 20 wt. %;

ratio of talc powder to the foaming material: 0.01:1.
(6) Alunite

pharmacological effects: germproofing, agglutination, treating eczema and pruritus, stopping itch;

pathological effects: detoxification, destroying vermifuge, drying dampness, stopping itch;

chemical components: KAl₃(SO₄)₂(OH)₆;

content in the herbs: 15 wt. %;

ratio of alunite to the foaming material: 0.0075:1.
(7) Cnidium Monnieri(L.)Cuss

pharmacological effects: anti-fungus, anti-Gram(−) bacteria, mildew-proofing, anti-dermatophyte;

pathological effects: dispelling wind, drying dampness, destroying vermifuge, stopping itch;

chemical components: L-piuene, L-camphene, bornyl, isovalerate, isoborneol, edultim, cnidimine, xanthotoxin;

content in the herbs: 10 wt. %;

ratio of Cnidium monnieri(L.)Cuss to the foaming material: 0.005:1.
(8) Hydnocarpus Anthelmintica P

pharmacological effects: germproofing, treating tenea pedis;

pathological effects: detoxification, destroying vermifuge, treating pruritus;

chemical components: chaulmoogric acid, hydnocarpic acid, gorlic acid;

content in the herbs: 15 wt. %;

ratio of hydnocarpus anthelmintica P to the foaming material: 0.0075:1.

By associating the Chinese herbaceous extracts according to the above formula with the plastic or rubber material through a blending process, articles with germproof and mildew-proof effects can be obtained.

### BRIEF DESCRIPTION OF THE DRAWINGS

FIG. 1 shows the process for preparing the Chinese herbaceous extract of the present invention.

FIG. 2 shows the process for producing articles with germproof and mildew-proof effects of the present invention.

FIGS. 3˜7 show the pictures (a)˜(e) taken in photoelectric viability tests of the produced article after a foaming treatment.

Attachment shows the results of germproof tests.

### DETAILED DESCRIPTION OF THE PREFERRED EMBODIMENTS

The foam with germproof and mildew-proof effects of the present invention is composed of Chinese herbaceous extracts and a plastic or rubber material and blended together through a foaming treatment. Accordingly, the articles made from such foam may possess germproof and mildew-proof effects. The herbs and additives used for preparing the Chinese herbaceous extracts of the present invention are preferably as follows:
(1) Angelica-Laxiflora:

pharmacological effects: relieving pain, reducing inflammation, mildew-proofing;

pathological effects: dispelling wind, drying dampness, relieving pain due to arthralgia, preventing dermatoses;

chemical components: angelica pubescens maxim, angelica acid angelol, angelicone, bergapten;

content in the herbs: 15 wt. %;

ratio of angelica-laxiflora to the foaming material: 0.0075:1.
(2) Ranunculacese-Clematis Chinensis

pharmacological effects: anodyne, stopping itch, treating pruritus;

pathological effects: dispelling wind, drying dampness, passing meridians, relieving pain;

chemical components: anemonin, hederagenin-anemonol, ponin (iresenin), sitosterin, myristic acid, linolenic acid, oleic acid;

content in the herbs: 12 wt. %;

ratio of ranunculacese-clematis chinensis to the foaming material: 0.006:1.
(3) Tripterygium Wilfordli Hook

pharmacological effects: anti-inflammation, immunosuppression;

pathological effects: reducing swelling or edema, relieving pain, treating pruritus, treating skin itch, treating skin spots, destroying vermifuge;

chemical components: wilfordine, witforgine, wilfortrine, tripdiolide, triptonide, hypolide, dulcitol;

content in the herbs: 10 wt. %;

ratio of tripterygium wilfordli hook to the foaming material: 0.005:1.
(4) Atractylodes Lancea(Thunb)DC

pharmacological effects: destroying bacteria, inhibiting growth of mold or fungus as formalin;

pathological effects: dispelling wind, drying dampness, inhibiting growth of bacteria, germproof, treating eczema, treating urticaria;

chemical components: atractylol, hinesol, β-eudesmol, β-selinene, acractylodine, 3,5-dimethoxy-lucosyloxy phenylpropenyl, alcohol;

content in the herbs: 15 wt. %;

ratio of Atractylodes lancea(Thunb)DC to the foaming material: 0.0075:1.
(5) Talc Powder

pharmacological effects: protecting mucous membranes of skin, absorbing chemical stimulants or toxicants;

pathological effects: treating eczema, treating acne;

chemical components: Mg₃(Si₄O₁₀(OH)₂)Al₂O₃;

content in the herbs: 20 wt. %;

ratio of talc powder to the foaming material: 0.01:1.
(6) Alunite

pharmacological effects: germproofing, agglutination, treating eczema and pruritus, stopping itch;

pathological effects: detoxification, destroying vermifuge, drying dampness, stopping itch;

chemical components: KAl₃(SO₄)₂(OH)₆;

content in the herbs: 15 wt. %;

ratio of alunite to the foaming material: 0.0075:1.
(7) Cnidium Monnieri(L.)Cuss

pharmacological effects: anti-fungus, anti-Gram(−) bacteria, mildew-proofing, anti-dermatophyte;

pathological effects: dispelling wind, drying dampness, destroying vermifuge, stopping itch;

chemical components: L-piuene, L-camphene, bornyl, isovalerate, isoborneol, edultim, cnidimine, xanthotoxin;

content in the herbs: 10 wt. %;

ratio of Cnidium monnieri(L.)Cuss to the foaming material: 0.005:1.
(8) Hydnocarpus Anthelmintica P

pharmacological effects: germproofing, treating tenea pedis;

pathological effects: detoxification, destroying vermifuge, treating pruritus;

chemical components: chaulmoogric acid, hydnocarpic acid, gorlic acid;

content in the herbs: 15 wt. %;

ratio of hydnocarpus anthelmintica P to the foaming material: 0.0075:1.

In the present invention, the foam is produced by integrally foaming a mixture containing 5 wt. % of herbaceous extracts and plastics or rubbers. Before foaming treatment, the above herbs are previously extracted and then ground with additives into powders. The additives can be talcs or alunite.

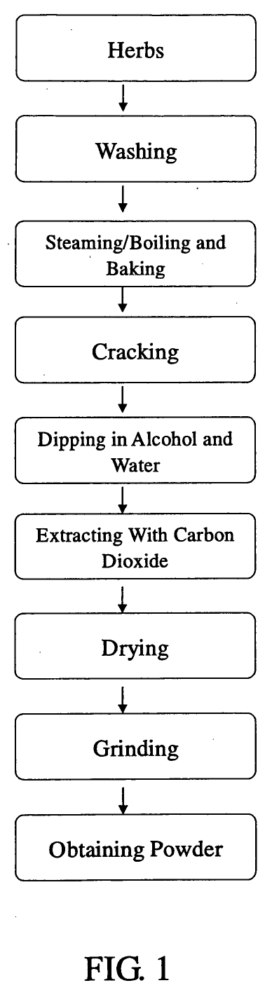

FIG. 1 shows a process for preparing the herbaceous extracts and includes steps of:

a) Washing the herbs with clean water, wherein the herbs include angelica-laxiflora, ranunculacese-clematis chinensis, tripterygium wilfordli hook, Atractylodes lancea(Thunb)DC, talc powder, alunite, Cnidium monnieri(L.)Cuss and hydnocarpus anthelmintica P;

b) Steaming/boiling the clean herbs at about 90° C.˜120° C., and then baking them at about 60° C.˜75° C. to destroy epidermis layers thereof; in which effective ingredients of the herbs are still preserved even in dry heat condition of about 170° C. and in wet heat condition of about 125° C.;

c) Cracking the baked herbs to obtain chippings in a size of about 80 mesh˜120 mesh;

d) Dipping the herbaceous chippings in a mixture containing alcohol and water for 24˜36 hours to active medicinal ingredients thereof, wherein a ratio of alcohol to water is 58%:42%, and a ratio of aqueous alcohol to the herbaceous chippings is 60%:40%;

e) Extracting the activated herbaceous chippings with carbon dioxide at a sub-critical temperature to isolate the activated ingredients;

f) Drying the extracts by either naturally drying at room temperature for 120˜240 hours or placing in a drier for five cycles wherein each includes drying for 3 hours at 60° C.˜75° C. and cooling to room temperature (25° C.) to prevent destruction from continuously heating; and thus the extracts can be further purified through this procedure;

g) Grinding the dried extracts with talc powder and alunite to obtain herbaceous powder with mildew-proof and germproof effects; in which the talc is a smooth silicate and the softest one among standard mineral in Mohs scale, and alunite is zinc sulfide.

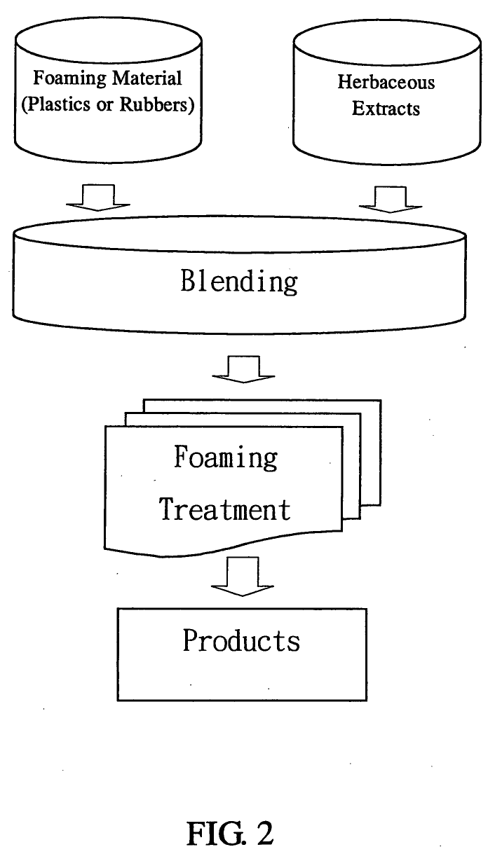

FIG. 2 indicates a process for blending the powdered extracts with the plastic or rubber material. In this process, the powdered extracts are completely blended with the plastic or rubber material in a container, and then brought to a foaming treatment below 125° C. to prevent destruction of the medicinal ingredients. According to such procedures, foam with Chinese herbaceous extracts can be obtained. The foam is suitable for manufacturing articles through proper processing treatments.

The plastic material aforementioned can be ethylene vinyl acetate (EVA) or polyethylene (PE), and the rubber material can be polyurethane (PU).

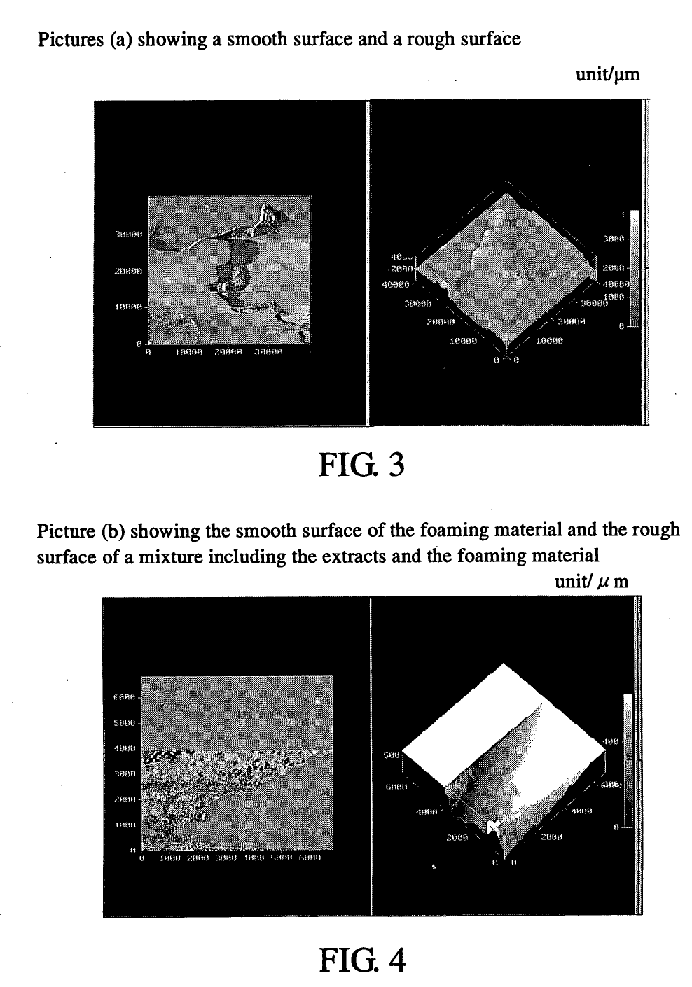

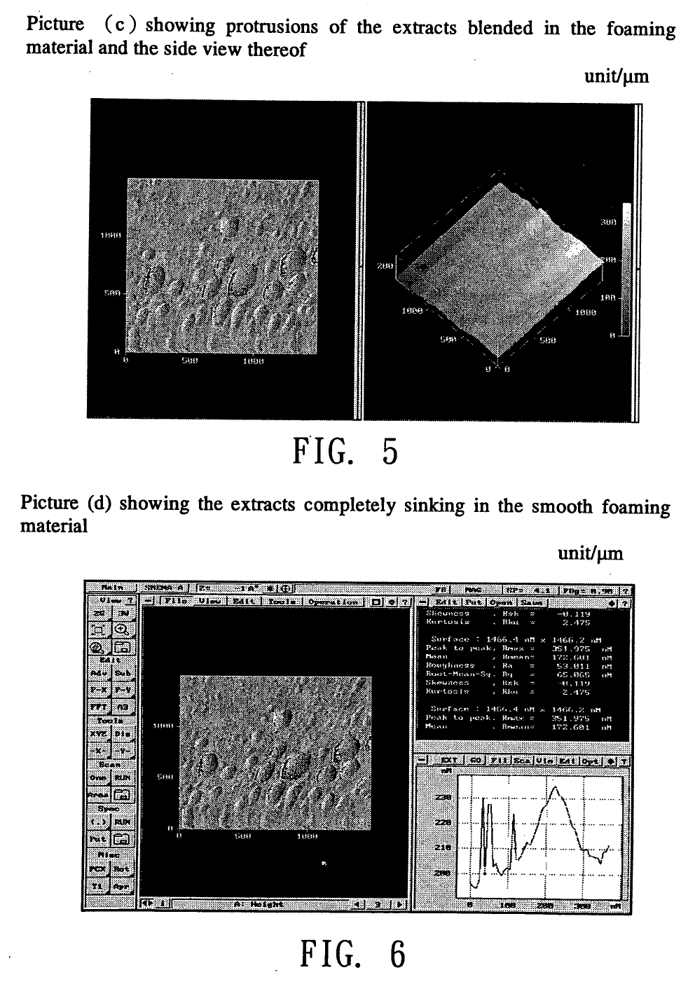

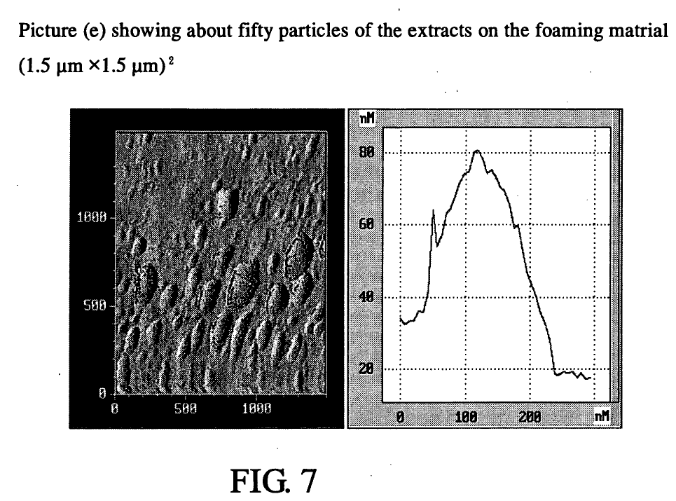

FIGS. 3˜7 show the pictures (a)˜(e) obtained in photoelectric viability tests of the produced article after the foaming treatment.

In FIG. 3, the right picture is perspective view of a cross section, and the left one is a top view of said cross section. That is, the cross section possesses both smooth and rough surfaces. In FIG. 4, the right picture is the rough surface of FIG. 3 and shows presence of the mixture including the extracts and the foaming material. The left picture is the smooth surface of FIG. 3 and shows the foaming material. In FIG. 5, the left picture is a side view of the right one and shows protrusions formed by the foaming material and the extracts enveloped therein. In FIG. 6, the curve shows the protrusions of FIG. 5. In FIG. 7, the picture shows at least fifty extracts particles presenting on the foaming material of an area 1.5 μm×1.5 μm.

Attachment shows the results of germproof tests, which is carried out by spraying a dispersion of the Chinese herbaceous extracts on a NA agar medium. The dispersion is prepared by dispersing the Chinese herbaceous extracts in sterile water. The cultures are then checked with several usual pathogens according to germproof tests, and the results indicate that the Chinese herbaceous extracts used in the present invention have excellent effect in inhibiting bacteria.

As the Chinese herbaceous extracts are natural and the plastic or rubber materials are synthetic polymer, no interaction occurs between them. Additionally, though the plastic or rubber material would become fatigue after using for a long time, the mildew-proof and germproof effects of the extracts can still last.

In the present invention, the Chinese herbaceous extracts with germproof and mildew-proof effects are applied to manufacturing articles by blending with the plastic or rubber material. Accordingly, appendant value of the daily-used articles is greatly promoted.

While the preferred embodiments of the invention have been described above, it will be recognized and understood that various modifications may be made therein and the appended claims are intended to cover all the modifications that may fall within the spirit and scope of the invention.

What is calimed is:

1. Foam with germproof and mildew-proof effects, comprising:
a plastic material; and
Chinese herbaceous extracts blended with said rubber material through a foaming treatment;
wherein said Chinese herbaceous extracts have germproof and mildew-proof effects.

2. The foam with germproof and mildew-proof effects as claimed in claim 1, which comprises at least 5 wt. % of said Chinese herbaceous extracts in the form of powder based on a total amount of said Chinese herbaceous extracts and said plastic material.

3. Foam with germproof and mildew-proof effects, comprising:
a rubber material; and
Chinese herbaceous extracts blended with said rubber material through a foaming treatment;
wherein said Chinese herbaceous extracts have germproof and mildew-proof effects.

4. The foam with germproof and mildew-proof effects as claimed in claim 3, which comprises at least 5 wt. % of said Chinese herbaceous extracts in the form of powder based on a total amount of said Chinese herbaceous extracts and said rubber material.

5. The foam with germproof and mildew-proof effects as claimed in claim 1, wherein said Chinese herbaceous extracts are obtained by treating herbs comprising about 15 wt. % of angelica-laxiflora, about 12 wt. % of ranunculacese-clematis chinensis, about 10 wt. % of tripterygium wilfordli hook, about 15 wt. % of Atractylodes lancea(Thunb)DC, about 10 wt. % of Cnidium monnieri(L.)Cuss and about 15 wt. % of hydnocarpus anthelmintica P, through steps of a) washing, b) steaming/boiling and baking, c) cracking, d) dipping, e) extracting, f) drying, and g) grinding with about 20 wt. % of talc powder and about 15 wt. % of alunite.

6. The foam with germproof and mildew-proof effects as claimed in claim 5, wherein said herbs are steamed or boiled at about 95° C.˜102° C. and then baked in a drier at about 95° C.˜102° C.

7. The foam with germproof and mildew-proof effects as claimed in claim 5, wherein said herbs are cracked to obtain chippings in a size of about 80 mesh˜120 mesh.

8. The foam with germproof and mildew-proof effects as claimed in claim 5, wherein said herbs are dipped in a mixture containing alcohol and water for 24˜36 hours, wherein a ratio of alcohol to water is 58%:42%, and a ratio of aqueous alcohol to the herbaceous chippings is 60%:40%.

9. The foam with germproof and mildew-proof effects as claimed in claim 5, wherein said herbs are extracted with carbon dioxide and then dried naturally at room temperature for 120˜240 hours.

10. The foam with germproof and mildew-proof effects as claimed in claim 5, wherein said herbs are extracted with carbon dioxide and then dried in a drier for five cycles wherein each includes drying for 3 hours at 60° C.˜75° C. and cooling to room temperature (25° C.).

11. The foam with germproof and mildew-proof effects as claimed in claim 1, wherein said plastic material is ethylene vinyl acetate (EVA).

12. The foam with germproof and mildew-proof effects as claimed in claim 1, wherein said plastic material is polyethylene (PE).

13. The foam with germproof and mildew-proof effects as claimed in claim 3, wherein said rubber material is polyurethane (PU).

14. The foam with germproof and mildew-proof effects as claimed in claim 3, wherein said Chinese herbaceous extracts are obtained by treating herbs comprising about 15 wt. % of angelica-laxiflora, about 12 wt. % of ranunculacese-clematis chinensis, about 10 wt. % of tripterygium wilfordli hook, about 15 wt. % of Atractylodes lancea(Thunb)DC, about 10 wt. % of Cnidium monnieri(L.)Cuss and about 15 wt. % of hydnocarpus anthelmintica P, through steps of a) washing, b) steaming/boiling and baking, c) cracking, d) dipping, e) extracting, f) drying, and g) grinding with about 20 wt. % of talc powder and about 15 wt. % of alunite.

15. The foam with germproof and mildew-proof effects as claimed in claim 14, wherein wherein said herbs are steamed or boiled at about 95° C.˜102° C. and then baked in a drier at about 95° C.˜102° C.

16. The foam with germproof and mildew-proof effects as claimed in claim 14, wherein said herbs are cracked to obtain chippings in a size of about 80 mesh˜120 mesh.

17. The foam with germproof and mildew-proof effects as claimed in claim 14, wherein said herbs are dipped in a mixture containing alcohol and water for 24˜36 hours, wherein a ratio of alcohol to water is 58%:42%, and a ratio of aqueous alcohol to the herbaceous chippings is 60%:40%.

18. The foam with germproof and mildew-proof effects as claimed in claim 14, wherein said herbs are extracted with carbon dioxide and then dried naturally at room temperature for 120˜240 hours.

19. The foam with germproof and mildew-proof effects as claimed in claim 14, wherein said herbs are extracted with carbon dioxide and then dried in a drier for five cycles wherein each includes drying for 3 hours at 60° C.˜75° C. and cooling to room temperature (25° C.).
